(19) United States
(12) Patent Application Publication
(10) Pub. No.: US 2022/0233775 A1
(43) Pub. Date: Jul. 28, 2022

(54) WEARABLE AUTO-INJECTOR DEVICES AND METHODS

(71) Applicant: Alexander Hassan, Ann Arbor, MI (US)

(72) Inventor: Alexander Hassan, Ann Arbor, MI (US)

(21) Appl. No.: 17/035,989

(22) Filed: Sep. 29, 2020

(51) Int. Cl. A61M 5/20; A61M 5/50

(52) U.S. Cl. CPC A61M 5/2033; A61M 5/50; A61M 5/204; A61M 2205/8206; A61M 2205/276; A61M 2205/3584; A61M 2205/58; A61M 2005/208; A61M 2230/04; A61M 2230/50; A61M 2205/3553; A61M 2210/08; A61M 2205/502

(57) ABSTRACT

Wearable auto-injector devices, systems and methods prevent medication administration in a device-worn state using a mechanism that prevents administration medication or by making it impossible or operationally impractical to physical orient or align the device to achieve injection. The device includes a housing, an attachment component enabling the housing to be worn by a user, and a medication delivery system including a needle and a medication reservoir. An advancement system advances the needle into a user's skin and causes the medication to flow through the needle. Various advancement systems, error-reduction mechanisms, attachment alternatives, and optional monitoring and communications capabilities are disclosed.

### FIELD OF THE INVENTION

This invention relates generally to injection devices for use in delivering medication to a user; more specifically to wearable auto-injectors and methods of using wearable auto-injectors.

### BACKGROUND OF THE INVENTION

Auto-injectors are a tool used by many to deliver therapeutic medication to users suffering from critical medical events, such as delivery of epinephrine to arrest severe allergic reactions or anaphylaxis, or thrombolytic medications in case of heart attack. Auto-injectors are designed to support “auto-” or “self-” injection by making it easier for a user to administer the medication than using a traditional syringe.

Related to the field of allergy, approximately 1 in 5 people suffer from allergies in the U.S. today, and many of those affected struggle with severe allergies, for which an untreated allergic response can lead to anaphylaxis or even a swift death. Sufferers of moderate to severe allergies are instructed to carry a dosage of epinephrine on their person at all times. The most common device for epinephrine distribution is Mylan's auto-injector, the EpiPen®, of which 50 million units have been distributed over the past 25 years.¹ The EpiPen® uses a hand-grip design, in which for use, the user's hand fully surrounds the device, presses the device against the target tissue, and injects the epinephrine. Kaleo's Auvi-Q® employs a similar design. ¹ https://www.epipen.com/personal-stories

While auto-injectors have been lifesaving tools for countless cases worldwide, current designs have several drawbacks. For one, the allergic patient must carry the device with them at all times, which is often impractical, due to the size and shape of the device. A 2018 study of epinephrine auto-injector (EAI) owners found that 45% of survey participants did not employ an epinephrine auto-injector at the time of severe reaction because an EAI was not available. Only 44% of participants claimed to carry an EAI on their person “all the time.” Indeed, 34% of children and adolescents reported even carrying an EAI on their person habitually.² ² Warren, C. M., Zaslaysky, J. M., Kan, K., Spergel, J. M., & Gupta, R. S. (2018). Epinephrine auto-injector carriage and use practices among US children, adolescents, and adults. Annals of Allergy, Asthma & Immunology. doi:10.1016/j.anai.2018.06.010.

Almost all prior art regarding traditional auto-injector designs describe mechanisms that would be highly non-obvious to decompose and redesign such that it is included in a wearable device. Most delivery systems, such as Mylan's EpiPen®, are highly elongated structures, in which the needle, needle safety, medication reservoir, plunger, dispensing mechanism, lockout mechanisms, and activation mechanisms are collinear along the longitudinal axis of the device and thus cause highly elongated device designs. The length of the device has been included as part of the delivery workflow, as the patient is expected to wrap their hand around the main body of the device (i.e. grasping the device with his/her hand in a ‘fist’ shape) and pushing the device into the target tissue. This has become the understood standard of care for auto-injectors.

While the concept of improving access to auto-injectors at time of critical medical event is underexplored, some have proposed solutions in this area. Students at Kent State University developed a solution in 2017 that integrated the delivery system into a smartphone case.³ Others have proposed generic “wearable” solutions, but are silent on how these non-specific solutions would be worn on the body, and critically, make no mention of designing similarity to, mimicry of, attachability to, or inclusion of functions of accessories that are already worn or carried by patients, such as the timekeeping characteristics of a wristwatch. Patients are hesitant to add additional burden to their routine; this is evidenced by the poor compliance in keeping EAIs on one's person. ³ https://www.allergicliving.com/2017/08/22/student-inventors-create-smartphone-case-that-administers-epinephrine/

In other proposals, the device is intended to deliver its medication in its wearable configuration, i.e. while being worn. This imposes challenges on the device design such as limiting the location of the wearable to specific locations on the body that do not support seamless integration into a user's lifestyle, and add concern for users that they may accidentally activate the device and inject themselves at an undesired time. As examples, US2017/0182242A1 and WO2016064266A2 make it clear that the delivery of the therapeutic is only performed in the wearable state. Another example is U.S. Pat. No. 9,180,244B2, which describes a wearable device intended to deliver medication (1) in a wearable state and (2) “by subcutaneous injection at slow, controlled injection rates”, the latter of which is ill suited for the delivery emergency therapeutics, such as epinephrine.

Other proposals have described concepts that are wearable on one's person and undergo a structural state change in the main housing in order to be able to deliver the medication. US2019/0209780A1 describes foldable components and hinges that enable significant elongation of the delivery assembly, in order to more closely mimic typical autoinjector designs (e.g. EpiPen®), which compromise the ability to blend into existing accessories and complicates the workflow for patient delivery.

### SUMMARY OF THE INVENTION

This invention is directed to wearable auto-injector devices, systems and methods. A device for injecting a medication constructed in accordance with the invention includes a housing, an attachment component enabling the housing to be worn by a user, and a medication delivery system disposed within the housing. The delivery system includes a needle and a medication reservoir in fluid communication with the needle. An advancement system, disposed within the housing, is operative to advance the needle through a user's skin and cause the medication to flow from the reservoir and through the needle for injection of the medication.

The device manually transitions from a worn state, wherein the attachment component is fully secured to the housing, to an unworn state, wherein at least a portion of the attachment component is detached from the housing to facilitate administration of the medication. The medication may epinephrine, administered to treat allergic reactions. However, those of skill in the relevant art will appreciate that the devices, systems and methods disclosed herein are applicable to other medications used to treat other conditions. Other medications that may be delivered include, but are not limited to, antiarrhythmic medication (such as during a cardiac emergency, e.g. a heart attack), thrombolytic medication (such as during a vascular emergency, e.g. a stroke), drug overdose reversal medication (such as the use of naloxone or nalmefene during an opiate or other addictive-substance overdose), or during a toxic exposure event (such as during a chemical or biologic exposure event, e.g. nerve agent, organophostate, anthrax, or sarin).

In the preferred embodiments of the invention, the medication cannot be administered when the device is in the worn state. To accomplish this, the device may further include a mechanism disposed within the housing that prevents the medication from being administered in the worn state. Alternatively, or in conjunction with such a mechanism, it may be impossible or overtly impractical to physical orient or align the device to achieve injection in the worn state.

The attachment component may be a strap adapted to encircle a user's wrist or other body part. Other configurations include wearing the device around: one's waist, in which the device mimics the behavior and appearance of a belt, one's neck, in which the device mimics the behavior and appearance of a necklace, or ones ankle, in which the device mimics the behavior and appearance of an ankle bracelet. Regardless, the attachment component may be unsecured by separating at least a portion of the attachment component from the housing.

The advancement system may include one or more compressed springs that are released to advance the needle or cause the medication to flow. Alternatively, the advancement system may use a compressed gas to advance the needle or cause the medication to flow. As yet a further alternative, the advancement system may include a battery-operated motor or other electromechanical unit to advance the needle or cause the medication to flow. The advancement system uses a multi-step activation sequence, or may be triggered by urging the device against tissue or by way of manual manipulation of the device.

The device may further include at least one mechanism for preventing exposure of the needle until use. Such a mechanism, which may comprise a moveable shield, effectively prevents needle exposure in the worn state. The device may further include apparatus for limiting premature activation of the advancement system. Such apparatus may include a manually operated mechanism, or may operate in conjunction with a sensor for detecting a specific condition. The device may further comprise apparatus that places the device into a non-injectable state after activation of the advancement system. The reservoir may be refillable, with or without removing the reservoir from the housing, and one or more internal components may be removable and/or replaceable.

At least one visual, audible or tactile indicator may be included to inform a user as to an operational status of the device. The housing may further include a clock or other timekeeping element; physiological monitor(s); GPS geolocating, and/or sensor(s) operative to detect a biomarker. Apparatus for wired or wireless communication with an external or remote device may also be integrated into the device. The device may be configured to record sensor or operational data, events, and/or communicate with a smartphone and/or directly with emergency personnel for various reasons. The device may further comprising one or more visual, audible or tactile alerts to indicate if the medication will expire or become compromised due to environmental factors.

The device of claim 1, wherein the attachment component is secured to an existing wrist, ankle or neck worn wearable accessory, and instructions for use of the device are located on the attachment component.

### BRIEF DESCRIPTION OF THE DRAWINGS

FIG. 1A is a simplified block diagram used to illustrate fundamental components of the invention;

FIG. 1B shows how components of FIG. 1A may be duplicated;

FIG. 1C illustrates on form of component sharing within a common housing;

FIG. 1D shows the way in which multiple reservoirs may share a common needle, advancement system and lock-out/retraction mechanism;

FIG. 1E illustrates the use of a single reservoir with multiple chambers that may or may not be refillable;

FIG. 1F is an isometric view of an embodiment of device 10 comprised of a housing and an attachment component;

FIG. 2 is a cross-sectional, top-down view of a preferred embodiment of the invention;

FIG. 3 is an exploded view with a top cover being shown as a separate component, which can be separated from the housing during normal use or limited to separation for assembly purposes;

FIG. 4 is a further isometric view wherein the top portion of the housing is hidden, thus exposing the internal components;

FIG. 5 is a diagram of optional surface features of the housing;

FIG. 6 shows a mechanism to put a device into a safe state after activation;

FIG. 7 shows how an electromechanical device may control the advancement of a plunger;

FIG. 8 shows how the movement of a shield may be governed by a rotary user input;

FIG. 9 is an isometric view of lateral and posterior device aspects;

FIG. 10 is an isometric view of a wearable auto-injector device, wherein instructions for use are located on an attachment component;

FIG. 11 is an isometric view of a wearable auto-injector device in conjunction with a conventional wristwatch;

FIG. 12 is an exploded view of a wearable device;

FIG. 13 illustrates a smartphone communicating with emergency personnel in conjunction with hardware and/or software integrated into any of the embodiments disclosed herein;

FIG. 14 is a workflow (or flowchart) applicable to embodiments of the invention wherein pressing a shield against target tissue causes exposure of the needle and enablement of an activation mechanism;

FIG. 15 is a workflow applicable to embodiments wherein squeezing the lateral aspect(s) of the housing allows the shield to translate relative to the needle;

FIG. 16 shows a workflow associated with the way in which the device may trigger a signal to the processor, which in turn interprets the signal and triggers communication with emergency personnel;

FIG. 17 illustrates a method of transitioning the device out of the worn configuration, locating the device at the target tissue, defeating one or more safety mechanisms, activating the device, and removing the device from the target tissue.

FIG. 18 is the workflow associated with detecting a temperature change in ambient environment;

FIG. 19 is a workflow wherein changes in the state of the device cause an indication to transition from invisible to a user to visible to a user; and

FIG. 20 is a workflow that shows reaching a predetermined end date.

### DETAILED DESCRIPTION OF THE INVENTION

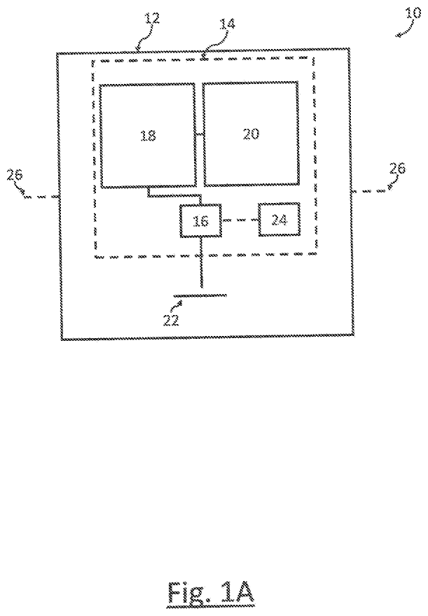

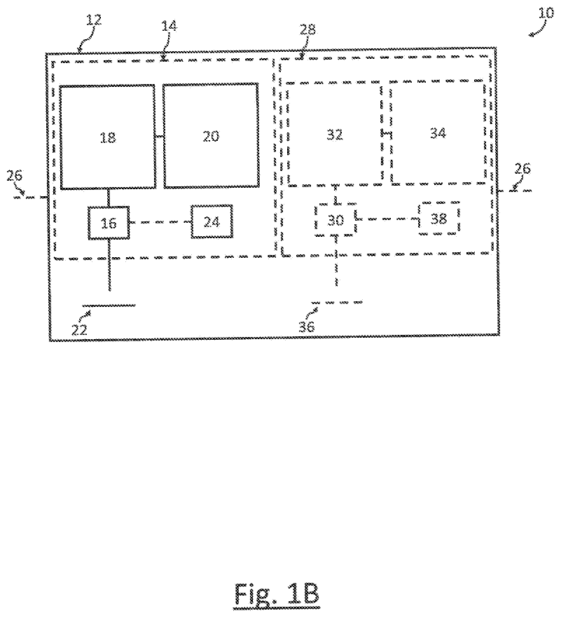

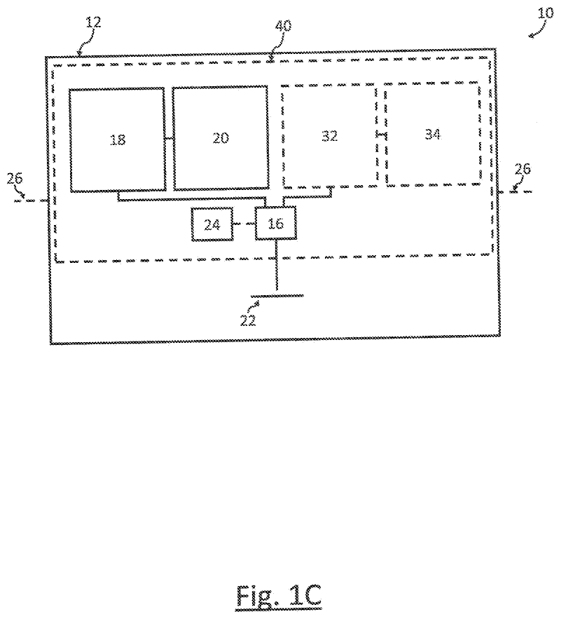

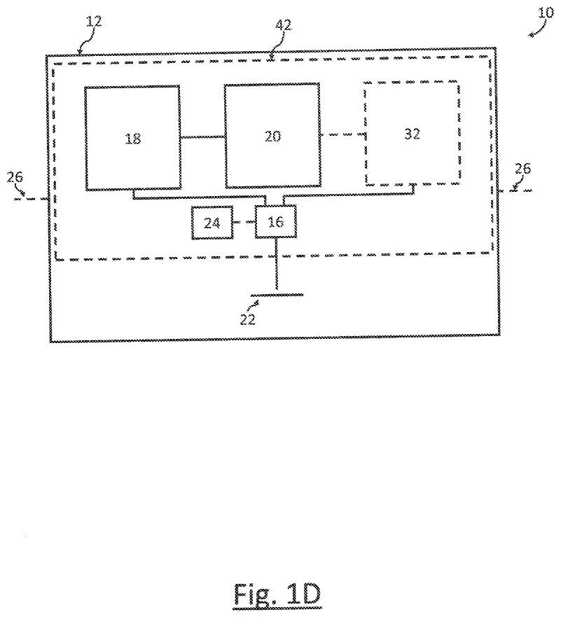

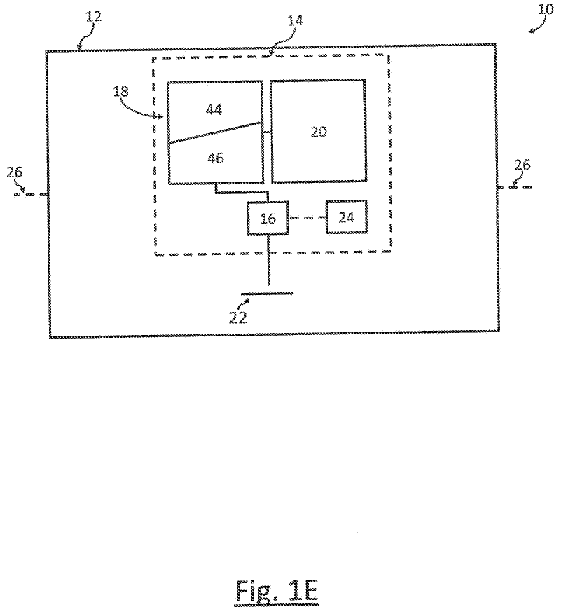

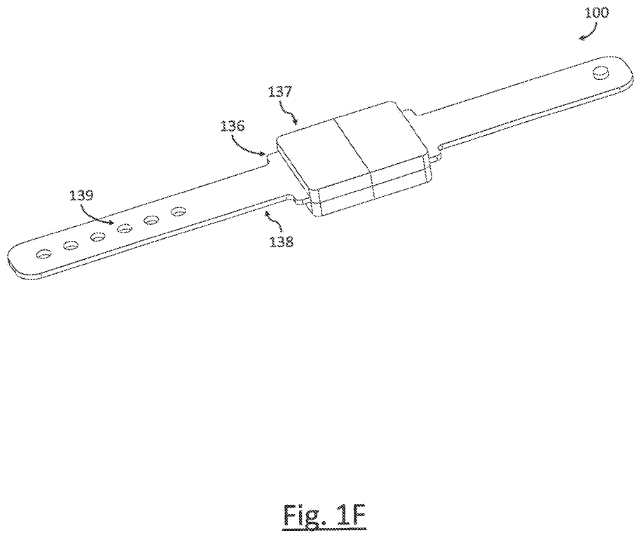

By way of introduction, FIG. 1A is a simplified block diagram that may be used to illustrate preferred embodiments of the invention. Device 10 includes an external housing 12 which contains a delivery system 14 that is comprised of a needle 16 for delivering a medication, a reservoir 18 connected to the needle which contains the medication, and an advancement system 20 for pushing the medication from the reservoir into the needle and into a patient. Multiple embodiments for the advancement system are described below. The device may optionally include a shield mechanism 22 for keeping the needle hidden when the device is not being used to deliver medication, but which allows the needle to deliver medication when desired, and a lock-out or retraction mechanism 24 which prevents re-deployment of the needle after the medication has been delivered. A fixed or removable attachment component 26 connects to the external housing, and is used to secure the wearable device to the user.

A user transitions the device from a ‘worn’ state, wherein the device is physically coupled to the user, to an ‘unworn’ state, wherein device is detached. In the ‘worn’ state, the device will have a limited ability to deliver the therapeutic medication. This limitation may be an explicit preventative measure built into the device, such as a lockout mechanism, or an implicit limitation, such as the difficulty in aligning and impaling the skin when the device is in a worn configuration.

In a preferred embodiment, the device 10 is worn on the wrist, similar to a conventional wristwatch. In this embodiment, the housing 12 may resemble the main body of a watch, with a strap similar to a watch strap. The device may also function as a watch or other body-worn device, powered by mechanical and/or electrical components and/or readouts. Such multi-functional characteristics of the device further increase the likelihood that users will keep it on their person at all times. As such, the device may include one or more of: the following features: timekeeping, heart-rate monitor, blood-pressure monitoring, pedometer, GPS, pulsometer, surface electromyography (EMG), other surface-based biomarker detection, or other features available on marketed smartwatches. In one embodiment, the device contains Bluetooth, RFID, or other near-field communication technology. In a sub-embodiment, this technology is used to communicate with a smartphone, and can be used to record and visualize the data being tracked by the sensors disclosed herein, including activation and/or delivery-of-medication events.

The advancement system 20 may be driven by springs that are released from a compressed or extended state. Alternatively, the advancement system 20 may be comprised of a mechanism to increase pressure in the reservoir 18, thus causing advancement of the medication into the patient. For example, a plunger may be advanced into the reservoir, thus pushing the medication into the patient. The advancement mechanism 20 may release a compressed fluid into a cavity immediately proximal to a plunger, thereby advancing the plunger toward the patient. As a further alternative, the advancement system 20 may be electro-mechanically powered, such as by a motor and battery system enclosed in the housing 12. In these and other embodiments, one of multiple mechanisms can be employed to “prime” or allow subsequent activation of the advancement system. This may be carried out by pressing the device against a target tissue, in which case a reaction force on the device causes activation. Activation may also be caused by input by a user's hand, such as pressing on a button or twisting a dial.

One or more safety mechanisms can be employed to prevent premature activation of the advancement system. As one example, activation may be mechanically prevented until the device is pressed against the target tissue. In another embodiment, activation may be mechanically prevented until an attachment component (e.g. strap) is separated from the housing by the user. In embodiments wherein the activation is performed by a motor, electrical signals may be used as a safety mechanism. In certain embodiments, the safety mechanism can be defeated by digital detection of the target tissue. That is, the device may prevent advancement of the needle or activation of the delivery system until the tissue is detected by one or more of: temperature, proximity, capacitance, optical and/or resistance sensors. For example, capacitive sensors on the surface of the device could signal contact with tissue and thereby allow for activation of the motor by the activation mechanisms described above. The device may use any type of sensor to identify presence or proximity of the tissue; the listed sensor types are intended to be illustrative and non-limiting.

Physical alignment, including rotational motion may be used for ensuring that the medication cannot be delivered prior to the intended time of delivery. For example, rotation of a user input mechanism on the housing could cause rotation of the reservoir so as to align the longitudinal axis of the needle and reservoir, and thereby allow communication between the two. Alternatively, rotation of a user input mechanism on the housing could cause rotation of a valve that governs communication between the reservoir and the needle and/or the reservoir and the activation mechanism.

If a shield mechanism 12 is provided, retraction thereof to keep the needle hidden until the user is ready to deliver the medication can be triggered by one or more of: the safety mechanisms described above or alternative mechanisms. Indeed, the device 10 may contain multiple safety mechanisms that must be defeated in order to advance the needle, deliver the medication, and/or retract the needle. These mechanisms may be dependent on one another. For example, the user may need to press the device against the target tissue before the user is able to squeeze a mechanical button, which in turn must be done before the user is able to press a button to trigger the activation mechanism.

The device 10 may include a mechanism to put the device into a safe state after the medication has been delivered. This may be caused by moving the needle or the needle and reservoir assembly away from the target tissue after completion of delivery. In mechanical embodiments, this may be effected by a compressed or extended spring that is held in place until the plunger can complete its full stroke (which corresponds with complete delivery of the medication). In embodiments where the delivery is activated by a motor, the retraction of the needle, needle and reservoir assembly, or needle and reservoir and plunger assembly may be effected by the motor.

Device re-usability is possible in accordance with the invention. The device 10 may be designed to be refilled by adding medication to the reservoir 18 after depletion. This may be accomplished using a separate port, conduit or syringe system to access the reservoir and inject the new medication. As an alternative, the device may be designed to support exchange of one or more parts of the delivery system, such as the needle, reservoir, and/or plunger. If the advancement mechanism 10 is powered by the release of compressed fluid, re-use could include replacement of the compressed fluid cartridge.

Ensuring proper use of the device is essential in life-threatening emergencies such as those that are targeted by this device. Providing clear instructions for use is critical in order to support reliable and repeatable use. Instructions for use may be located on the housing 12 or attachment component, such as a wrist strap. Clear communication to the user that the device is in a primed, activated, or completed state is important for correct use as well. An indicator on the device may change in response to the transition between states. For example, an indicator may change when the device transitions from a typical ‘worn’ state, to a ‘ready to deliver’ state, to a ‘delivering medication’ state, and/or to a ‘delivery complete’ state. The indicator may be visual, audible, or tactile. In embodiments in which the indicator is visual, this could include text, color change, or a mechanical change in the exterior housing. In embodiments where the indicator is audible, this could be caused by a mechanical trigger, such as an audible ‘click’, or electronic, such as with a battery and speaker system. In embodiments where the indicator is tactile, this could be caused by a mechanical trigger, such as a tactile ‘click’, or electronic, such as with a battery and haptic vibration system.

The device 10 may further be configured to automatically contact emergency personnel when it is activated via an integrated communication device. The device may accomplish this through wireless or wired connections, as via connection with a smartphone or other device.

Medication to be stored in the reservoir 18 typically has a limited duration of efficacy; after the expiration date, the medication is no longer certified for use. All medical devices of this type are required to have an expiration date listed on the packaging. The device 10 may therefore include one or more additional indicators to communicate that the contained medication is past its expiration date. The indicator(s) may be of any of the types of indicators listed throughout this application.

Medication to be stored in the reservoir 18, such as epinephrine, is subject to accelerated degradation associated with environmental factors, such as temperature. This is critical information for users, as the concentration of the needed medication can decline substantially, such as when an epinephrine auto-injector is stored in a car during summer months. The device may therefore include an indicator to communicate that environmental factors may have compromised the therapeutic. A change in this indicator may be caused by the device being exposed to temperatures at or above (or at or below) a specific temperature for a duration of time. As a point of reference, some marketed autoinjectors for epinephrine have a maximum recommended storage temperature of 25° C. An example of this feature in this device could be a material that undergoes a color change when it is heated above 25° C. for a prolonged period of time. The indicator(s) may be of any of the types of indicators listed throughout this application.

All of the device characteristics described herein may also be applied to a device that attaches to an existing accessory, rather than being a standalone device. For example, the device 10 may be an attachment to a conventional wristwatch, in which the external housing and delivery system are affixed to the wristwatch, and utilize the existing attachment mechanism (e.g. wrist strap) to remain accessibly located on the patient. In this embodiment, the detachment of the housing and delivery system from the conventional wristwatch could serve similar safety functions to the detachment from the strap in the preferred embodiment (in which all aspects are integrated in a single device). Note that “wristwatch” should be taken to include “smart” watches, and “existing accessory” may be taken to include other body-worn objects such as necklaces and bands or straps that at least partially encircle extremities or other body parts.

Note further that internal components maybe duplicated in accordance with different embodiments of the invention. FIGS. 1B through 1E show alternate internal configurations of the invention, in which the device is configured to enable multiple deliveries of medication to a patient. FIG. 1B is a simplified block diagram to describe an alternate internal configuration of the invention, which contains multiple delivery systems. The device 10 is again shown with housing 12 and attachment component 26, and has a first delivery system 14 and a second delivery system 28. The first delivery system 14 is identical to as shown in FIG. 1A. The second delivery system 28 mirrors the first delivery system 14, comprising a second needle 30 for delivery a medication, a second reservoir 32 connected to the needle which contains the medication, a second advancement system 34 for pushing the medication from the reservoir into the needle and into a patient, and a second lockout or retraction mechanism 38. In FIG. 1B, the second delivery system 28 is shown with a second shield mechanism 36, though shield mechanism 22 may keep both needles hidden, as opposed to having second shield 36.

FIG. 1C is a simplified block diagram of another alternate internal configuration, which contains a single delivery system 40, comprising the components from the delivery system 14 as shown in FIG. 1A (needle 16, reservoir 18, advancement system 20, and lockout or retraction mechanism 24), a second reservoir 32, which is connected to needle 16, and a second advancement system 34. In this embodiment, the device 10 uses a single needle 16, shield 22, and lockout or retraction mechanism 24, and needle 16 is in communication with one or more reservoirs.

In configurations with multiple advancement systems, such as those shown in FIG. 1B and 1C, the second advancement system 34 may be configured to prevent advancement until the first advancement system 20 has been used. FIG. 1D is a simplified block diagram of another alternate internal configuration, which contains a single delivery system 42, comprising the components from the original delivery system 14 (needle 16, reservoir 18, advancement system 20, and lockout or retraction mechanism 24), a second reservoir 32, which is connected to needle 16, and is connected to advancement system 20. In this embodiment, advancement system 20 is configured advance medication from the first reservoir 18 upon first activation, and to advance medication from the second reservoir 32 upon second activation. It is apparent by one skilled in the art that this single-advancement system configuration may also be used with a first and second needle, as is shown in FIG. 1B.

FIG. 1E is a simplified block diagram of another alternate internal configuration, which contains the delivery system 14, shows a configuration comprising the components from the delivery system 14 as shown in FIG. 1A (needle 16, reservoir 18, advancement system 20, and lockout or retraction mechanism 24), but where the delivery system is configured to deliver a portion of the medication contained in the reservoir as part of a first activation of advancement system 20, and is configured to deliver another portion of the medication contained in the reservoir as part of a second activation of advancement system 20.

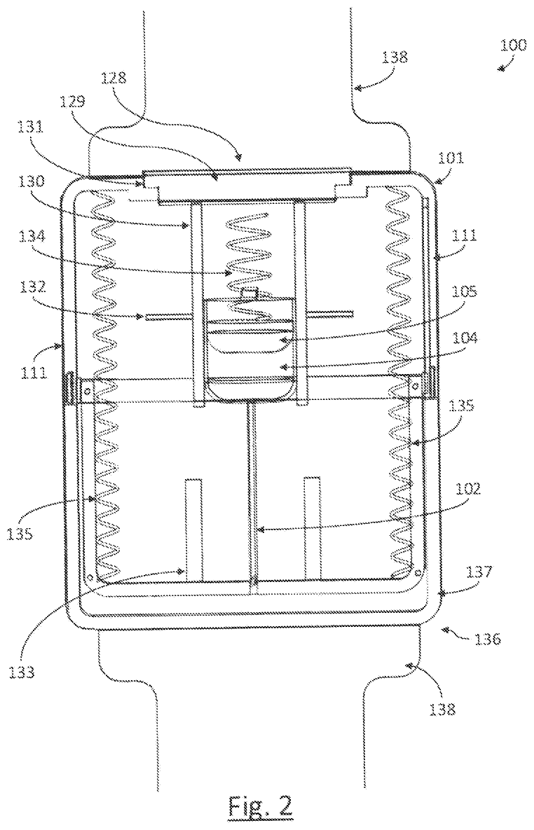

FIG. 2 provides a cross-sectional top-down view of a preferred embodiment of the invention, depicted generally at 100. For the purposes of this figure, the words upward, downward, and lateral will be used in reference to the orientation of the image, but it should be noted that the device does not necessarily need to be used in an orientation corresponding to upward and downward relative to gravity. The outer housing 101 is shown supporting a reservoir 104 and needle 102. A shield 103 covers the needle. A plunger 105 is shown partially translated within the reservoir. The attachment component 136 and its rigid member 137 which connects to the housing are shown.

An activation button 129 is shown above plunger 105, with two elongate members 130 on either side of the reservoir. A spring 134 is shown connected to the plunger 105. This spring 134 constraints the distance between the activation button and the plunger 105. In the embodiment shown, the elongate members 130 are biased inward, and are only forced to the outside of the reservoir due to the mechanical interference between the member 130 and the reservoir 104. Without the presence of the reservoir, the members 130 would move inward toward the vertical longitudinal axis of the device.

When the device 100 is to be used, the user removes the strap 136 thus exposing the bottom aspect of the shield 103. The bottom aspect of shield 103 is pressed against the target tissue when the device is to be used. Shield 103 translates upward along slots 111, thereby exposing the needle 102. Springs 135 resist this upward motion and ensure that the exposure of the needle is intentional. Shield 103 has two elongate members 133 as well. As the shield translates upward, the shield's elongate members 133 contact the activation button's elongate members 130, forcing them upward. Once the members 130 are no longer forced outward by the lateral aspects of the reservoir 104, they move inward and rest above the top of the plunger 105. The user then presses down on the activation button 129, causing a downward force on the plunger 105, thus propelling the medication in reservoir 104 into the needle 102 and into the user. Other features shown in FIG. 2 include a step-down component 131 on the activation button that prevents the button from moving inside the outside walls of the housing 101, and a physical stopper 132 that prevents the button from sliding completely outside the housing as it is forced upward.

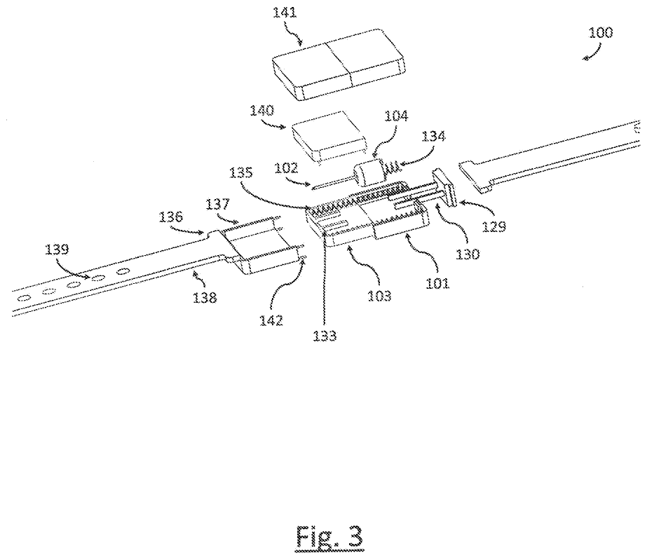

FIG. 3 shows an exploded view of device 100. In this embodiment, the top cover 141 is shown as a separate component, which can be separated from the housing 101 during normal use or limited to separation for assembly only. The shield 103 is also shown as a separable component, with a top element 140 and a bottom element 103. Alignment features are shown on 140 to ensure proper alignment between 140 and 103, and should be assumed to be optionally present on any other components of the device in which external surfaces are aligned. Alignment features 142 are also shown between the rigid member of the strap 137 and the housing 101. These features 142 may optionally prevent movement of the shield 103 or activation button 129 until they are separated from the housing 101. Features such as alignment structure(s) 142 can be applied to any advancement and activation mechanism, including embodiments that are motorized or driven by compressed fluid.

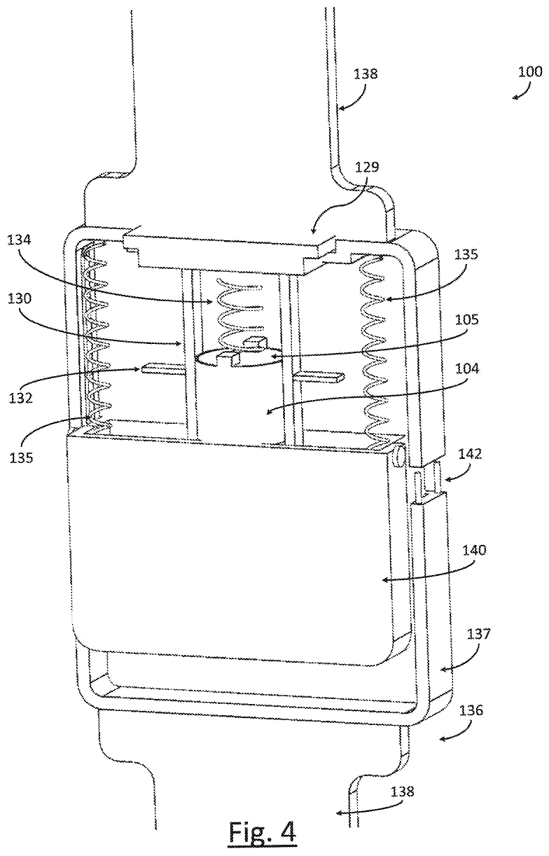

FIG. 4 shows another isometric view of the device 100. In this Figure, the top housing is hidden, thus exposing the internal components. In this figure, the top cover of the shield 140 is shown attached to the bottom of the shield 103.

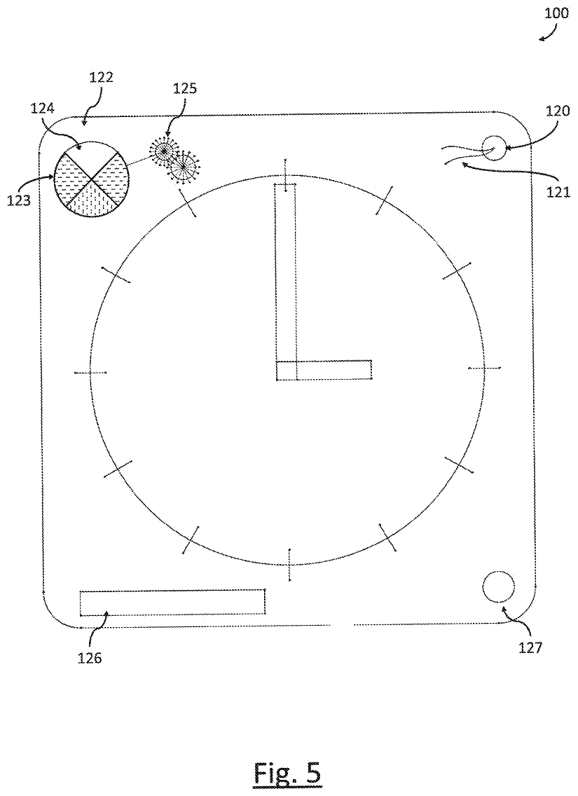

FIG. 5 is a diagram of optional surface features of the housing, including device 100. More or fewer of these surface features may be present on any embodiment. A timekeeping element 119 is shown as the main indicator on the surface of the device. An LED 120 is shown, with wires 121 that can connect to elements internal to the device, such as a motor, computing processor, or battery. A visual indicator assembly 122 is shown, with a radial element 123 with distinct sections, and a window 124, arranged such that the user may only see one section of the radial element 123 at a time. A set of gears 125 that optionally connect to the internal mechanisms of the device are shown. The radial element 123 may be moved by physical motion of internal components, including translation of the shield or activation button, pressure change such as the release of a compressed fluid, or by a motor and battery system. Rather than radial element 123 the window 124 may be moveable, due to one or more of the same physical motion changes. A digital display 126 is shown, and may be connected to a computing processor and battery. An unpowered visual indicator is shown in 127, and can be used to communicate changes in environmental conditions. For example, the visual indicator 127 may be a thermochromic ink that changes color in response to exceeding a prespecified temperature for a certain amount of time, thus communicating that the medication may be compromised. In another embodiment, visual indicator 127 may change color after exposure to oxygen for a certain amount of time, thus communicating that the medication has expired.

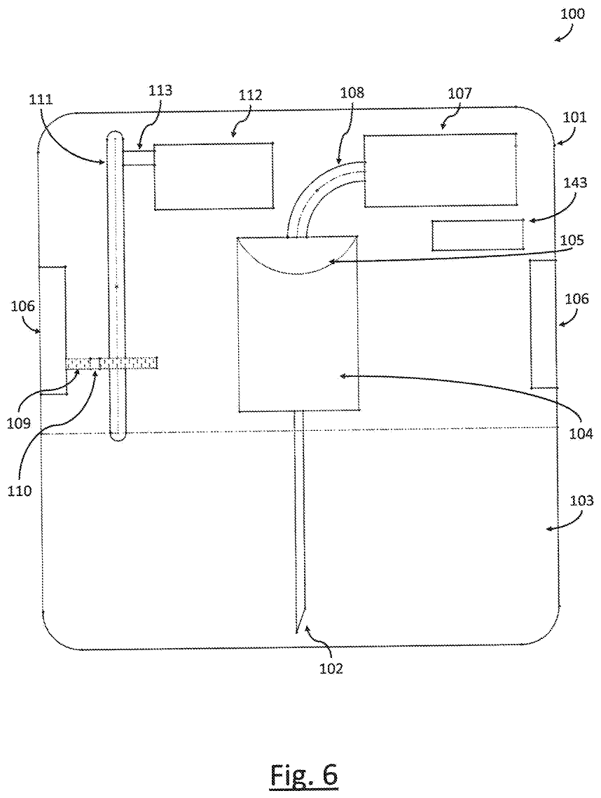

FIG. 6 shows an embodiment of device 100 wherein the advancement of the plunger 105 is driven by the release of a compressed gas. A compressed fluid storage chamber 107 is shown, with a communication 108 between the chamber 107 and the plunger 105. This figure also shows mechanical elements for squeezing 106 on the lateral aspects of the housing 101. A mechanism 109 is shown wherein the user squeezes the lateral touchpoints 106 inward, thus moving a primary rail 109 inward, thereby aligning an opening in the rail 110 to align with the slot 111 on which the shield translates. Therefore, in this embodiment, a user needs to squeeze the lateral aspects of the device in order to expose the needle 102. The safety mechanism afforded by 109 and 110 is only shown on one side of the device but can optionally be employed on both sides of the device. The safety mechanism may also be used to limit motion of the activation mechanism, such as the plunger 105 or an activation button as shown in other figures.

FIG. 6 also shows a mechanism to put the device 100 into a safe state after activation. A second chamber with a compressed fluid 112 is connected to the slot along with the shield 103 translates. Following activation of the device, 112 releases fluid into slot 111 via the physical communication 113, thereby forcing the shield to re-cover the needle 102 and prevent reactivation of the device. FIG. 6 also shows an area 143 that can house one or more elements, including a battery, speaker system, wireless communication system, or one or more secondary functional elements, such as a heart-rate monitor, pedometer, pulsometer, electromyography (EMG) sensor, Global Positioning System (GPS) sensor, and sensor to detect biomarkers on the skin. Depending on the content of area 143, these elements may be connected to a computer processor within area 143 or a separate area of the device. In some embodiments, these elements may be triggered by mechanical motion of other aspects of the device, in which that motion allows for completion of a circuit thus triggering a function of one or more of the elements. For example, motion of the plunger 105 may trigger an electrical contact between two wires, thus completing a circuit between a battery and speaker system, so as to trigger an audible alert when the plunger 105 has moved.

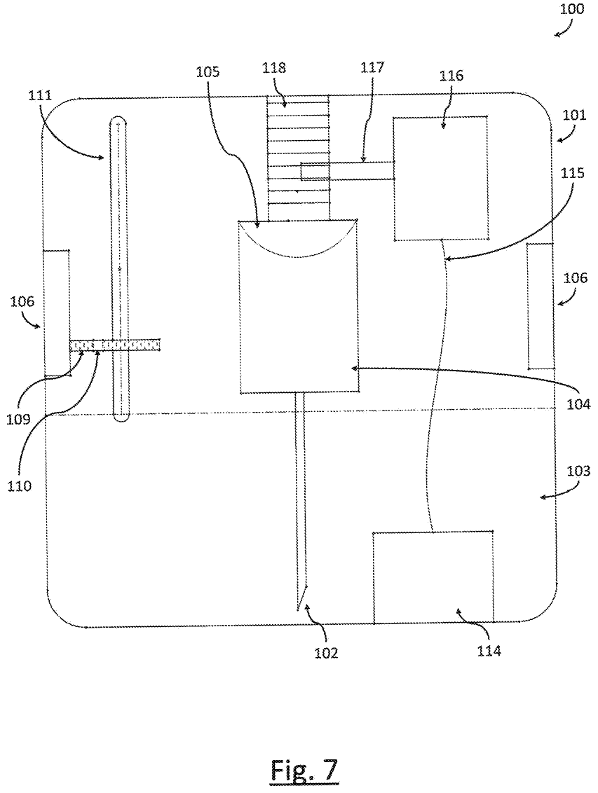

FIG. 7 shows how the advancement of the plunger 105 may be controlled by an electromechanical device such as a linear actuator such as a solenoid or rotational drive such as a motor system, which may include stepper motors. In this figure, a motor 116 is shown connected to a main shaft 117, which is connected to a main body 118 connected to the plunger 105. The main shaft 117 has a gearing element on its distal tip, and the main body 118 has recipient teeth, such that upon spinning motion of the shaft 117 by the motor 116, the main body 118 moves downward, propelling the medication out of the reservoir 104, into the needle 102 and into the user. The area shown for the motor 116 may also include a battery and computer processing system, such as a microcontroller. This figure also shows a sensor 114 on the bottom aspect of the shield 103, as well as a connection between the sensor and the motor assembly 115. One or more sensors may be in the device; one is shown in this figure for illustrative purposes. Sensors may include, but are not limited to, temperature, proximity, capacitance, or resistance sensors. The area shown for the sensor 114 may also include a computer processor. Once the sensor 114 has detected certain conditions, the device may then trigger activation of the motor, thus triggering advancement of the medication.

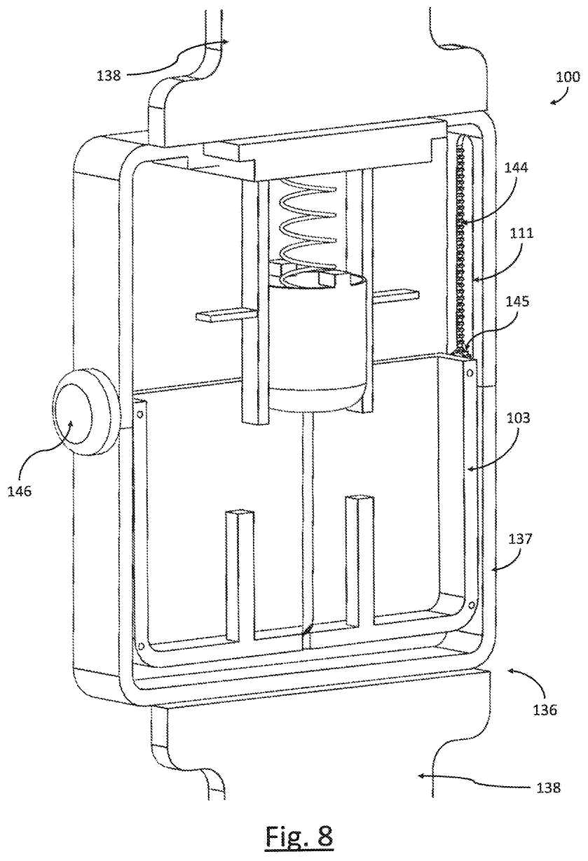

FIG. 8 shows how the movement of the shield 103 may be governed by a rotary user input. This embodiment maintains a removable attachment component 136, comprised of rigid member 137 and flexible member 138. A rotary input dial 146 is shown on the lateral aspect of the housing. Rotation of this dial causes upward motion of a geared or toothed member 145 (located on shield 103), within slot 111, along a matching rack interface 144. The rigid member of the attachment component 137 can optionally be used to prevent rotation of the dial until it [rigid member 137] has been removed from the device. The rack and gear embodiment shown should be considered one of several possible rotary mechanisms, including screw mechanisms. The dial on the lateral face should be considered one of several possible locations, including on the top face of the device.

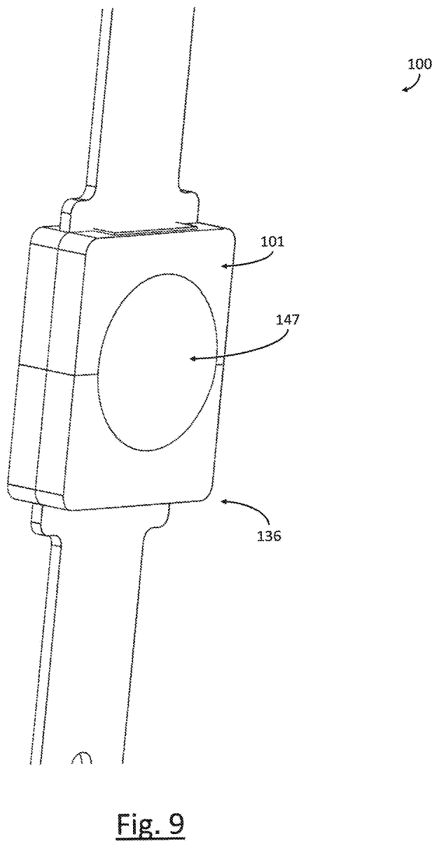

FIG. 9 is an isometric view of the lateral and posterior aspects of device 100. A sensor array 147 is shown on the posterior aspect of the device, such that this sensor array would be in contact with the user's skin in the ‘worn’ configuration. This sensor array may be attached to housing 101 or attachment component (i.e., strap) 136.

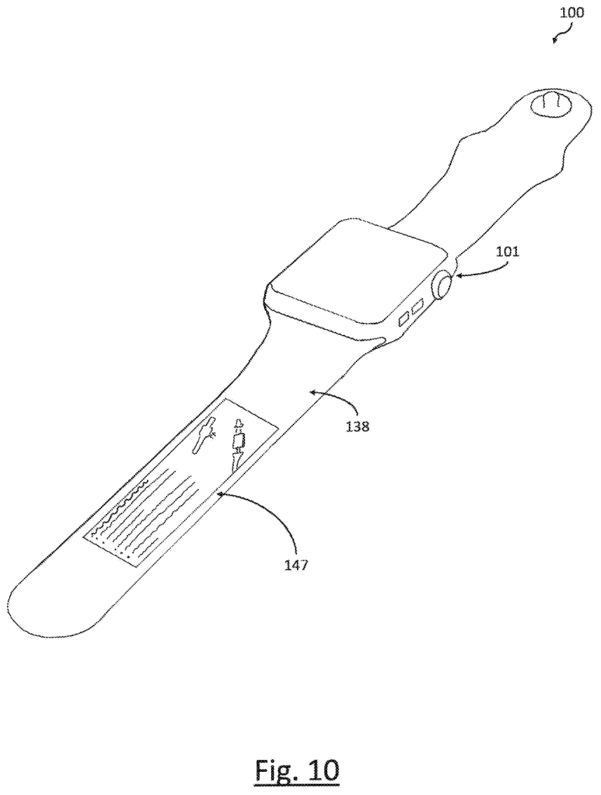

FIG. 10 is an isometric view of a wearable autoinjector device 100, wherein instructions for use 148 are located on the attachment component. In this figure, they are shown on flexible member 138.

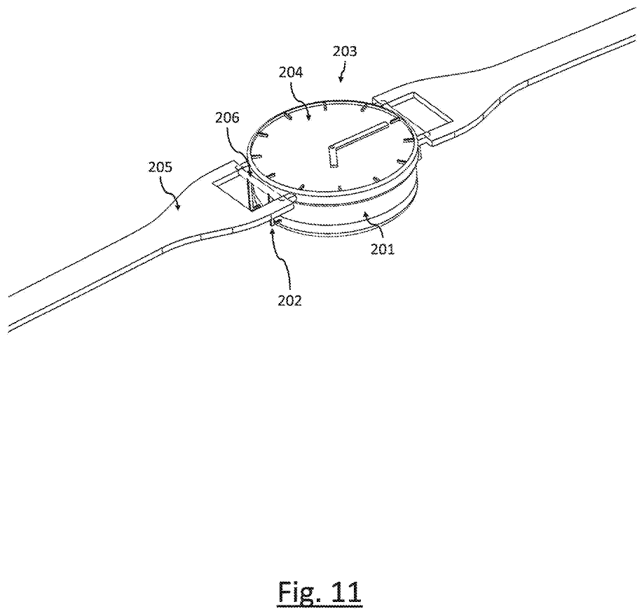

FIG. 11 is an isometric view of a wearable autoinjector device 201 in conjunction with a conventional wristwatch 203. The wearable device is shown connected at a junction between the main body of the wristwatch 204 and its strap 205 at a cylindrical interface 206, via links 202 that extend from the main body of device 201. It is to be noted that in this configuration, the device is not itself connected to the user via any attachment component; rather the attachment component (in this figure, shown by 202) is used to connect the device to a conventionally worn accessory, such as wristwatch 203.

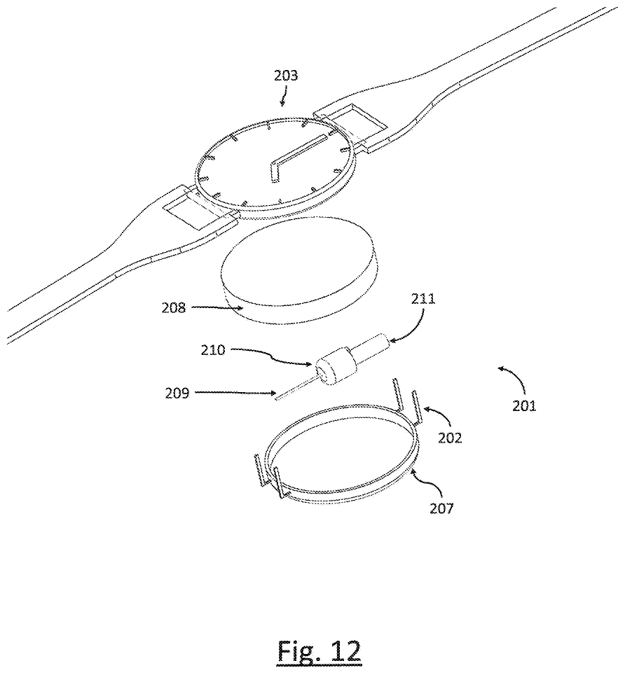

FIG. 12 shows an exploded view of the wearable device 201. In this figure, 201 is shown as being comprised of a lower housing 207, attachment component 202, an upper housing 208, a needle 209, reservoir 210, and plunger assembly 211. Other internal components of the device as described for embodiments in which the device attaches to the user, rather than a worn accessory, (such as shield, slot, activation button, etc.) should be assumed to apply to device 201.

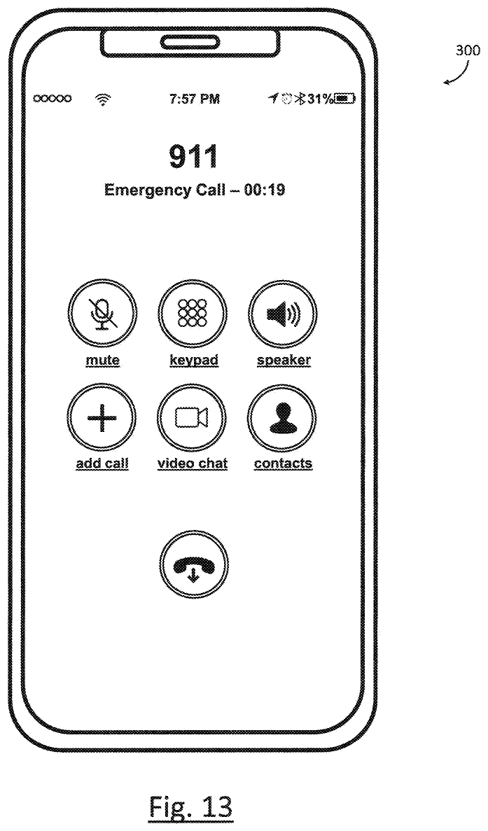

FIG. 13 shows a smartphone 300 communicating with emergency personnel in conjunction with hardware and/or software integrated into any of the embodiments disclosed herein. For example, device 100 or 201 can automatically trigger a response from smartphone 300 upon relevant transition states. It can communicate with smartphone 300 via a communication component, such as that shown by area 143 in FIG. 6.

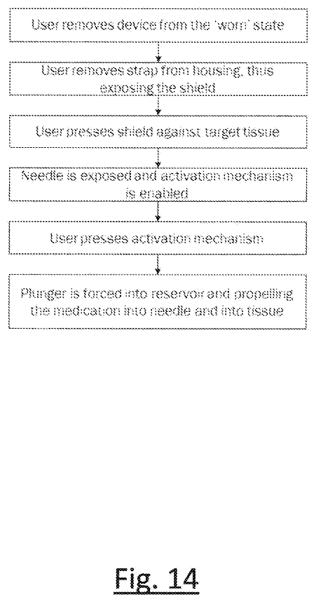

FIG. 14 shows a workflow applicable to embodiments wherein pressing the shield against target tissue causes exposure of the needle and enablement of the activation mechanism.

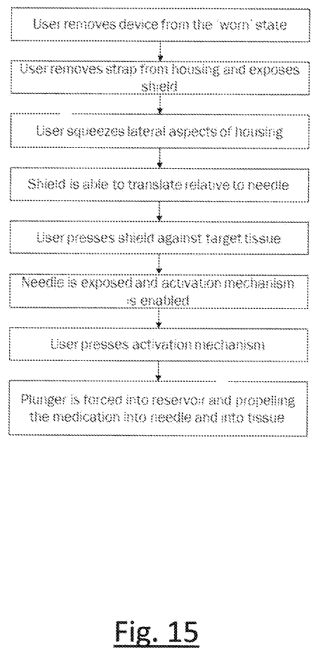

FIG. 15 shows a workflow applicable to embodiments wherein squeezing the lateral aspect of the housing allows the shield to translate relative to the needle.

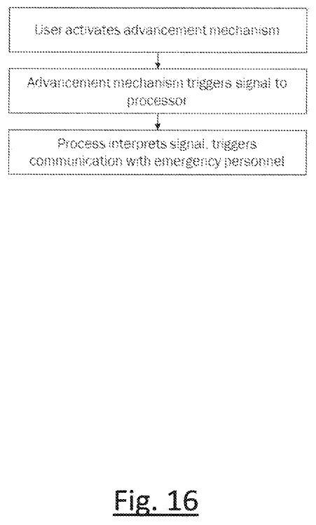

FIG. 16 shows a workflow associated with the way in which the device may trigger a signal to the processor, which in turn interprets the signal and triggers communication with emergency personnel.

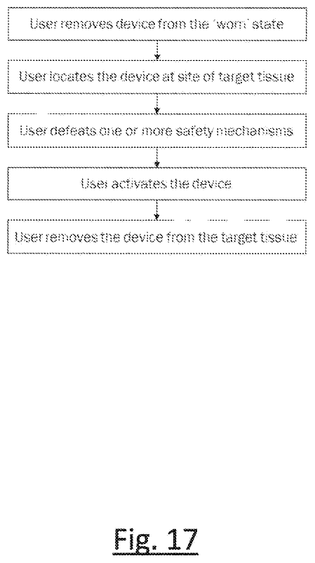

FIG. 17 illustrates a method of transitioning the device out of the worn configuration, locating the device at the target tissue, defeating one or more safety mechanisms, activating the device, and removing the device from the target tissue.

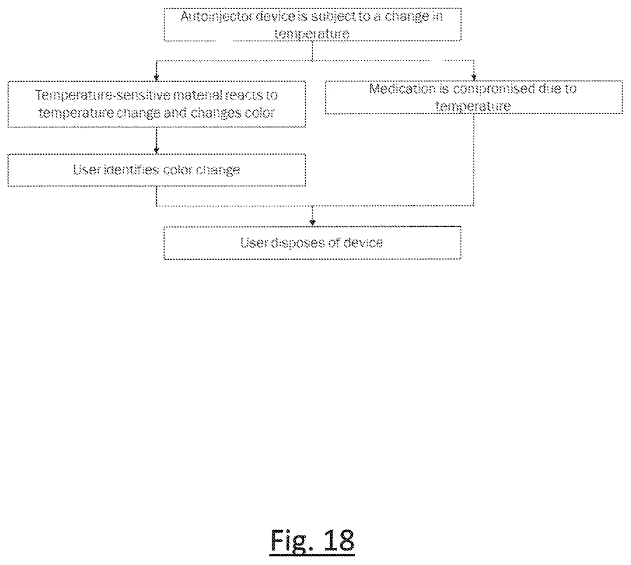

FIG. 18 shows the workflow associated with detecting a temperature change in the environment, wherein the temperature change compromises the integrity of the medication, in which the temperature change causes a state change in a material in or on the device, wherein that state change causes a visible color change, thus causing the user to dispose of the device.

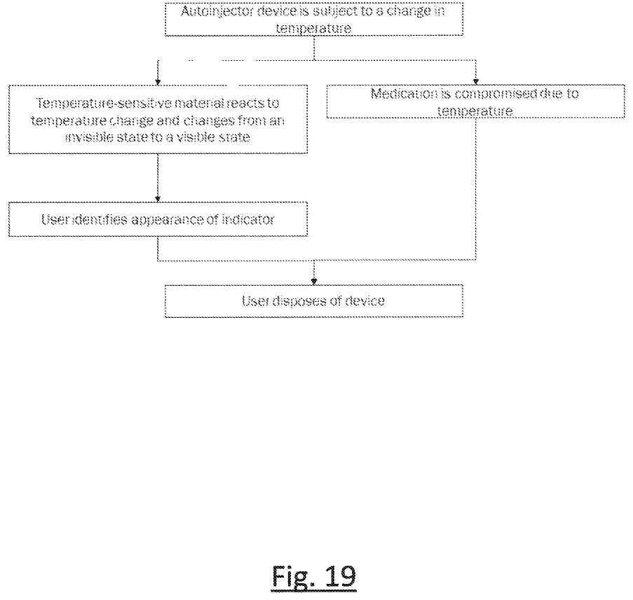

FIG. 19 shows a workflow wherein changes in the state of the device cause an indication to transition from invisible to a user to visible to a user.

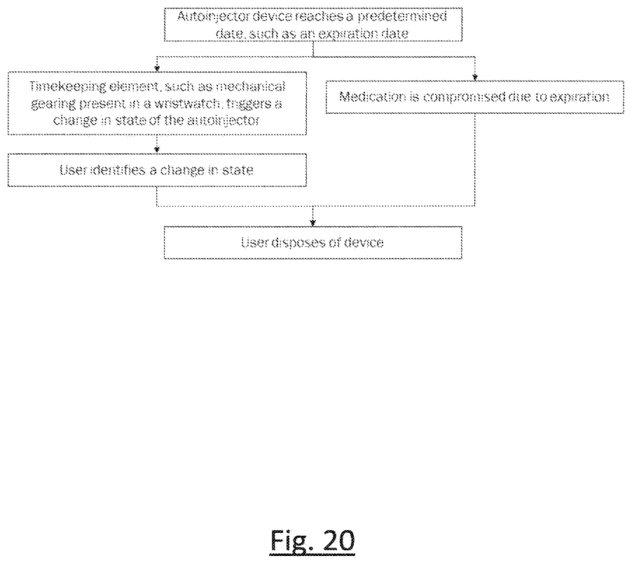

FIG. 20 shows a workflow of the device reaching a predetermined end date, at which time a timekeeping element triggers a state change, which is identified by the user, and thus causing the user to dispose of the device.

1. A device for injecting a medication, comprising:
a housing;
an attachment component enabling the housing to be worn by a user;
a medication delivery system disposed within the housing, the delivery system including a needle and a medication reservoir in fluid communication with the needle;
an advancement system disposed within the housing, the advancement system being operative to advance the needle through a user's skin and cause the medication to flow from the reservoir and through the needle for injection of the medication;
wherein the device transitions from a worn state, wherein the attachment component is fully secured to the housing, to an unworn state, wherein at least a portion of the attachment component is detached from the housing; and
wherein the medication cannot be administered in the worn state.

2. The device of claim 1, wherein the medication is epinephrine.

3. The device of claim 1, further including a mechanism disposed within the housing that prevents the medication from being administered in the worn state.

4. The device of claim 1, wherein the device cannot be physically oriented for injection in the worn state.

5. The device of claim 1, wherein the attachment component is a strap adapted to encircle a user's wrist or other body part.

6. The device of claim 1, wherein component is unsecured by separating at least a portion of the attachment component from the housing.

7. The device of claim 1, wherein the advancement system includes one or more compressed springs that are released to advance the needle or cause the medication to flow.

8. The device of claim 1, wherein the advancement system uses a compressed gas to advance the needle and cause the medication to flow.

9. The device of claim 1, wherein the advancement system includes a battery-operated motor or other electromechanical unit to advance the needle and cause the medication to flow.

10. The device of claim 1, wherein the advancement system uses a multi-step activation sequence.

11. The device of claim 1, wherein the advancement system is triggered by urging the device against tissue.

12. The device of claim 1, wherein the advancement system is triggered by through a manual manipulation of the housing.

13. The device of claim 1, further including at least one mechanism for preventing exposure of the needle until use.

14. The device of claim 13, wherein the mechanism is a moveable shield.

15. The device of claim 13, wherein the mechanism prevents exposure in the worn state.

16. The device of claim 1, further comprising apparatus for limiting premature activation of the advancement system.

17. The device of claim 16, wherein the apparatus for limiting premature activation of the advancement system is a manually operated mechanism.

18. The device of claim 16, wherein the apparatus for limiting premature activation of the advancement system includes a sensor for detecting a specific condition; and
further including a mechanism that prevents premature activation unless the specific condition is detected.

19. The device of claim 18, wherein the specific condition includes one or more of the following:
temperature,
proximity,
capacitance and
resistance.

20. The device of claim 1, further comprising apparatus that places the device into a non-injectable state after activation of the advancement system.

21. The device of claim 1, wherein the reservoir is refillable without removing the reservoir from the housing.

22. The device of claim 1, wherein one or more of the following are replaceable:
the needle;
the reservoir;
the advancement system; and
compressed gas.

23. The device of claim 1, further comprising at least one indicator that informs a user as to an operational status of the device.

24. The device of claim 23, wherein the operational status includes one or more of the following conditions:
the device is in worn state;
the device is ready or primed for use;
the device is delivering medication; and
medication delivery complete.

25. The device of claim 23, wherein the indicator is a visual, audible or tactile indicator.

26. The device of claim 1, further comprising one or more of the following also disposed within the housing:
a clock or other timekeeping element;
a heart-rate monitor;
pedometer;
pulsometer;
blood-pressure monitor;
electromyography (EMG) sensor;
Global Positioning System (GPS) sensor; and
a sensor operative to detect a biomarker.

27. The device of claim 1, further comprising apparatus for wired or wireless communication with an external or remote device.

28. The device of claim 27, wherein the wireless communication is performed using one or more of Bluetooth, RFID, near-field communication (NFC), and cellular communications.

29. The device of claim 1, wherein the device is configured to perform one or more of the following functions:
record sensor data, activation events or medication delivery events; and
communicate with emergency personnel when the device becomes ready for activation, delivers medication, or completes delivery of medication.

30. The device of claim 1, wherein the device is configured to communicate with a smartphone to perform one or more of the following functions:
record sensor data, activation events or medication delivery events; and
communicate with emergency personnel when the device becomes ready for activation, delivers medication, or completes delivery of medication.

31. The device of claim 1, further comprising one or more visual, audible or tactile alerts to indicate if the medication will expire or become compromised due to environmental factors.

32. The device of claim 1, wherein the attachment component is secured to an existing wearable accessory.

33. The device of claim 32, wherein the wearable accessory is wrist, ankle or neck worn.

34. The device of claim 1, wherein instructions for use of the device are located on the attachment component.
